(19) United States
(12) Patent Application Publication
(10) Pub. No.: US 2022/0142972 A1
(43) Pub. Date: May 12, 2022

(54) PARASITICIDAL FORMULATIONS AND USE THEREOF

(71) Applicant: Elanco US, Inc., Greenfield, IN (US)

(72) Inventor: Robert ZOLYNAS, Shawnee, KS (US)

(21) Appl. No.: 17/417,063

(22) PCT Filed: Dec. 19, 2019
(86) PCT No.: PCT/US19/67430

§ 371 Date: Jun. 21, 2021

Related U.S. Application Data
(60) Provisional application No. 62/783,434, filed on Dec. 21, 2018.

(51) Int. Cl. A61K 31/365; A61K 31/7048; A61K 31/05; A61K 47/10; A61K 9/00; A61K 47/22

(52) U.S. Cl. CPC A61K 31/365; A61K 31/7048; A61K 47/22; A61K 47/10; A61K 9/0017; A61K 31/05

(57) ABSTRACT

A formulation for the dermal control of endoparasites comprising a macrocyclic lactone and method of use thereof.

### CROSS REFERENCE TO RELATED APPLICATIONS

This application is an international application filed under the Patent Cooperation Treaty and claims priority to U.S. Provisional Patent Application No. 62/783,434, filed Dec. 21, 2018, the content of which is incorporated herein in its entirety.

### BACKGROUND

### 1. Field

The present invention relates to parasiticidal formulations and method for dermal control of endoparasites in animals.

### 2. Description of Related Art

There is an ongoing need for compositions and methods for control of endoparasites in animals. For example, gastrointestinal nematode infections of dogs and cats are of ongoing concern. In dogs, in most cases such infection is brought about by species of the three nematode families Ascarididae, Ancylostomatidae and Trichuridae. In cats, it is predominantly the two nematode families Ascarididae and Ancylostomatidae, which have spread worldwide. These infections, such as roundworms, hookworms and whipworms, cause considerable problems, especially in young, growing dogs, cats, and also in humans.

In addition to gastrointestinal nematode infections mentioned above, there are further severe nematode parasitoses, for example filiarioses, which are highly host-specific.

The parasite Dirofilaria immitis—a filarial endemic in parts of North to South America, Africa, Asia, and Australia, is the cause of the important canine and feline cardiovascular dirofilariasis (heartworm disease). The severe pathophysiological changes within the cardiovascular system which occur during the Dirofilaria immitis infection of dogs and cats can bring about a dramatic course of the disease in the host animal.

Although there are endoparasiticidal agents against gastrointestinal nematodes and other agents with an action against Dirofilaria immitis in dogs and cats, the effectiveness of known formulations is not always entirely satisfactory. Therefore, there is an ongoing need to provide methods and compositions to treat and control such endoparasites and nematodes in order both to cure animals already affected and to maintain as yet uninfected animals in a healthy condition.

A solution to this technical problem is provided by the compositions and methods of the present invention.

### BRIEF SUMMARY

The present application provides a parasiticidal formulation comprising about 0.1 to about 10% by weight of one or more macrocyclic lactones, about 60 to about 95% by weight of one or more solvents selected from the group consisting of benzyl alcohols and optionally substituted pyrrolidones, and about 5 to about 60% by weight of one or more cosolvents selected from the group consisting of cyclic carbonates and lactones, wherein the one or more macrocyclic lactones are the sole parasiticidal active compound.

The present application also relates to a parasiticidal formulation comprising about 0.5 to about 5% by weight of one or more macrocyclic lactones, about 80 to about 95% by weight of one or more solvents selected from the group consisting of benzyl alcohols and optionally substituted pyrrolidones, and about 10 to about 20% by weight of one or more cosolvents selected from the group consisting of cyclic carbonates and lactones.

Methods for controlling endoparasites in an animal comprising applying to the animal the parasiticidal formulation of the present invention are also provided.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

The present application relates to parasiticidal compositions which comprise a macrocyclic lactone, such as an avermectin, a 22,23-dihydroavermectin B₁ (ivermectin) or a milbemycin.

New formulations for the dermal application of macrocyclic lactones have now been found which are particularly suitable for dermal control of endoparasites, such as gastrointestinal nematodes (e.g., roundworm, hookworm, and whipworm) and cardiovascular nematodes (e.g., heartworm) in animals, such as humans, livestock, and pets.

The formulations according to the invention have the following composition:

macrocyclic lactones in a concentration of from 1 to 10% by weight based on the overall weight of the formulation;

solvents from the group of benzyl alcohol or optionally substituted pyrrolidones in a concentration of from 0 up to 95% by weight based on the overall weight of the formulation;

if desired, further solvents from the group consisting of cyclic carbonates or lactones in a concentration of from 5.0 up to 60% by weight based on the overall weight of the formulation;

if desired, further adjuvants from the group of thickeners, spreading agents, colorants, antioxidants, propellants, preservatives, adhesives, emulsifiers, in a concentration of from 0.01 up to 10% by weight based on the overall weight of the formulation.

Parasiticidal Active Compounds

The invention therefore relates to compositions including macrocyclic lactones, in particular, avermectins, B1 22,23-dihydroavermectins (ivermectins) or milbemycins.

Avermectins are macrolide lactone compounds or compound mixtures of the general formula (I)

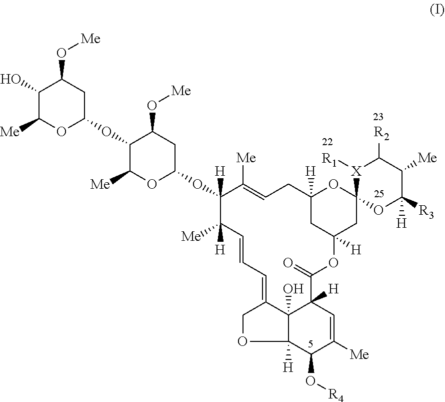

in which

the radicals R¹ to R⁴ can have the meaning give in Table 1 below and X can represent a single or double bond between the C₂₂ and C₂₃ positions (—C₂₂R¹—X—C₂₃R²—).

When the bond is double, there are no substituents (R¹, R²) at the C₂₂ and C₂₃ positions.

TABLE 1

| Macrocyclic lactone | —C₂₂R¹—X—C₂₃R²— | R³ | R⁴ |
|---|---|---|---|
| Avermectin A₁ₐ | —CH═CH— | -sec-Bu | —Me |
| Avermectin A_{1b} | —CH═CH— | -iso-Pr | —Me |
| Avermectin A₂ₐ | —CH₂—CHOH— | -sec-Bu | —Me |
| Avermectin A_{2b} | —CH₂—CHOH— | -iso-Pr | —Me |
| Avermectin B₁ₐ | —CH═CH— | -sec-Bu | —H |
| Avermectin B_{1b} | —CH═CH— | -iso-Pr | —H |
| Avermectin B₂ₐ | —CH₂—CHOH— | -sec-Bu | —H |
| Avermectin B_{2b} | —CH₂—CHOH— | -iso-Pr | —H |
| 22,23- | —CH₂—CH₂— | -sec-Bu | —H |
| dihydroavermectin B₁ₐ | | | |
| 22,23- | —CH₂—CH₂— | -iso-Pr | —H |
| dihydroavermectin B_{1b} | | | |
| Doramectin | —CH═CH— | -Chx | —H |

22,23-dihydroavennectin B₁ stands for ivermectin B₁;
sec-Bu = secondary butyl;
iso-Pr = isopropyl;
Chx = cyclohexyl;
—Me = methyl

The avermectins and B₁22,23-dihydroavermectins (ivermectins) of the general formula (I) are often used as mixtures. The product abamectin, which essentially contains the B₁ avermectins, and their hydrogenation products the B₁22,23-dihydroavermectins (ivermectin) are of particular interest in this connection.

Furthermore, the semisynthetic macrocyclic lactone selamectin (5-hydroxyimino-25-cyclohexylavermectin B₁ monosaccharide) is derived from the avermectins:

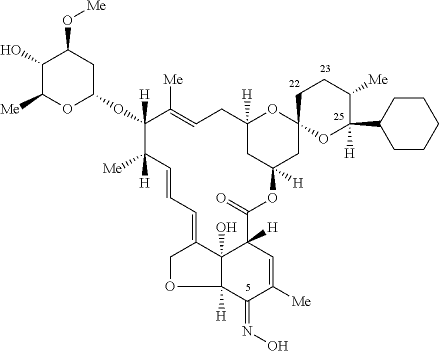 [SMILES: CO[C@H]1C[C@H](O[C@@H]2/C(C)=C/C[C@@H]3C[C@@H](C[C@]4(CC[C@H](C)[C@@H](C5CCCCC5)O4)O3)OC(=O)[C@@H]3C=C(C)/C(=N\O)[C@H]4OC/C(=C\C=C\[C@@H]2C)[C@]43O)O[C@@H](C)[C@@H]1O]

Eprinomectin ((4″R)-4″(acetylamino)-4″-deoxyavermectin B₁) is likewise derived from the avermectins; this term is understood as meaning a mixture of 90% or more of component B₁ₐ and 10% or less of component B_{1b}:

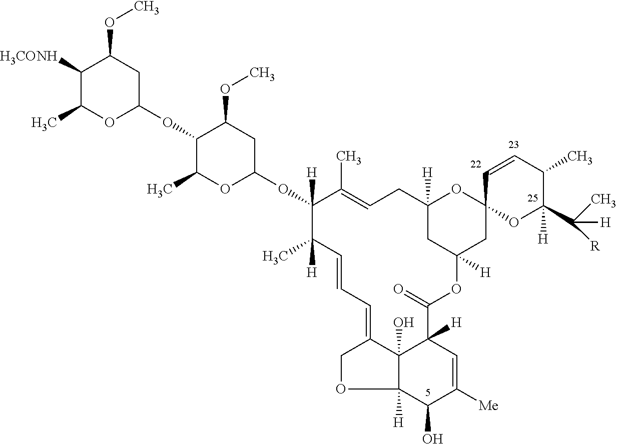

Component B₁ₐ: R=C₂H₅

Component B_{1b}: R=CH₃

While the milbemycins have the same macrolide ring structure as avermectins or B₁22,23-dihydroavermectins (ivermectins), they do not carry any substituent (i.e. missing oleandrose disaccharide fragment) at position 13 (R⁵=hydrogen).

Milbemycins from the class of macrocyclic lactones which may be mentioned by way of example are the compounds having the general formula (II)

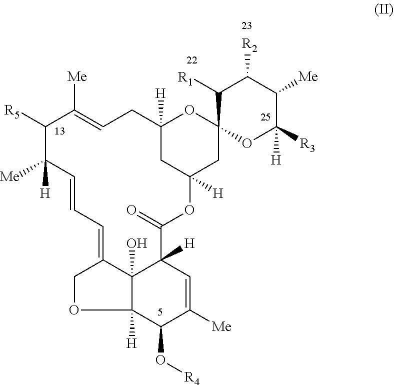

in which

the radicals R¹ to R⁵ have the meanings given in Table 2 below:

TABLE 2

| Macrocyclic | | | | | |
|---|---|---|---|---|---|
| lactone | R¹ | R² | R³ | R⁴ | R⁵ |
| milbemycin | —H | —H | -iso-Pr | —H | —H |
| B41 D | | | | | |
| nemadectin | —H | —OH | 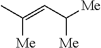 | —H | —H |
| moxidectin | —H | ═N—O—Me | 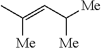 | —H | —H |

iso-Pr = isopropyl

In connection with the milbemycins, mention may also be made of milbemycin oxime, which is as a rule employed as a mixture of 80% milbemycin A₄5-oxime and 20% milbemycin A₃5-oxime:

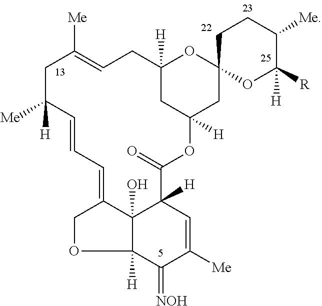

milbemycin A4 oxime: R=—CH2CH3

milbemycin A4 oxime: R=—CH3

Those of the above mentioned macrocyclic lactones which are of particular interest in accordance with the invention are:

avermectin B₁ₐ/B_{1b} (or abamectin)

22,23-dihydroavermectin (B1a/B1b) (or ivermectin B₁ₐ/B_{1b})

doramectin

moxidectin

selamectin

eprinomectin.

In some embodiments, the formulation contains an additional parasiticidal active compound, such as an ectoparasiticidal compound. Examples of ectoparasiticidal compounds include, but are not limited to, neonicotinoids (such as dinotefuran, imidacloprid, and nitenpyram), phenylpyrazoles (such as fipronil), organophosphates (such as chlorpyrifos, dichlorvos, and malathion), carbamates (such as carbaryl and propoxur), formamidines (such as amitraz), oxadiazines (such as indoxacarb), insect growth regulators (such as methoprene, fenoxycarb, and pyriproxyfen), pyrethrins and pyrethroids (such as permethrin, fluzenprox, and etofenprox), and spinosyns (such as spinosad).

In a preferred embodiment, one or more macrocyclic lactones are the sole parasiticidal compounds present in the formulations of the invention.

Within the meaning of the invention, the active substances are also understood as being their pharmaceutically acceptable salts, hydrates and prodrugs, insofar as they can be used.

The above-mentioned active substances can, where appropriate in dependence on the nature and number of the substituents, be present in the form of stereoisomers, e.g. geometric and/or optical isomers, or regioisomers, or in the form of corresponding isomeric mixtures of different composition. Both the pure isomers and the isomeric mixtures having a corresponding effect can be used in accordance with the invention.

The formulations according to the invention contain the active substance in concentrations of from 0.1 to 10% by weight, preferably from 0.5 to 5% by weight, more preferably, from 1 to 3% by weight.

In other embodiments, the formulations according to the invention contain the active substance in concentrations of about 0.1% by weight, about 0.25% by weight, about 0.5% by weight, about 0.75% by weight, about 1.0% by weight, about 1.25% by weight, about 1.5% by weight, about 1.75%, about 2.0% by weight, about 2.25% by weight, about 2.5% by weight, about 2.67% by weight, about 2.75% by weight, about 3.0% by weight, about 3.25% by weight, about 3.5% by weight, about 3.75% by weight, about 4.0% by weight, about 4.25% by weight, about 4.5% by weight, about 4.75% by weight, about 5.0% by weight, about 5.25% by weight, about 5.5% by weight, about 5.75% by weight, about 6.0% by weight, about 6.25% by weight, about 6.5% by weight, about 6.75% by weight, about 7.0% by weight, about 7.25% by weight, about 7.5% by weight, about 7.75% by weight, about 8.0% by weight, about 8.25% by weight, about 8.5% by weight, about 8.75% by weight, about 9.0% by weight, about 9.25% by weight, about 9.5% by weight, about 9.75% by weight, or about 10.0% by weight.

Preparations that are diluted before use contain the active substance in concentrations of from 0.5 to 90% by weight, preferably from 1 to 50% by weight.

In general, it has proved advantageous to administer quantities of from about 0.5 to about 50 mg, preferably from 1 to 20 mg, of active substance per kg body weight once-a-month in order to achieve effective results.

For example, 10 mg active substance may be administered to an animal (e.g., a dog) that weighs 3.0-9.0 lbs; 25 mg active substance may be administered to an animal (e.g., a dog) that weighs 9.1-20.0 lbs; 62.5 mg active substance may be administered to an animal (e.g., a dog) that weighs 20.1-55.0 lbs; 100 mg active substance may be administered to an animal (e.g., a dog) that weighs 55.1-88 lbs; 125 mg active substance may be administered to an animal (e.g., a dog) that weighs 88.1-110 lbs; etc.

The formulations can be administered to provide the desired results, for example, once a day, once a week, or advantageously once a month, or even less frequently, such as every two months. In some embodiments, such as for the treatment of an intestinal parasite, a single administration may be effective. In other embodiments, such as for the prevention of heartworm disease, repeated administrations (e.g., once-a-month administrations) are preferred.

Solvents

Suitable solvents include benzyl alcohol or optionally substituted pyrrolidones. Examples of optionally substituted pyrrolidones include 2-pyrrolidone, 1-(C₂₋₂₀-alkyl)-2-pyrrolidone, in particular 1-ethylpyrrolidone, 1-octylpyrrolidone, 1-dodecylpyrrolidone, 1-isopropylpyrrolidone, 1-(s- or t- or n-butyl)pyrrolidone, 1-hexylpyrrolidone, 1-(C₂₋₂₀-alkenyl)-2-pyrrolidone such as 1-vinyl-2-pyrrolidone, 1-(C₃₋₈-cycloalkyl)-2-pyrrolidone such as 1-cyclohexylpyrrolidone, 1-(C₁₋₆-hydroxyalkyl)-2-pyrrolidone, 1-(C₁₋₆-alkoxy-C₁₋₆-alkyl)-2-pyrrolidone such as 1-(2-hydroxyethyl)-pyrrolidone, 1-(3-hydroxypropyl)pyrrolidone, 1-(2-methoxyethyl)-pyrrolidone, 1-(3-methoxypropyl)pyrrolidone, and also 1-benzylpyrrolidone. Particular preference is given to benzyl alcohol or n-dodecyl- or n-octylpyrrolidone.

In other embodiments, solvents which have very good macrocyclic lactone-dissolving properties can be used in the formulation, such as ethanol, isopropanol, propylene glycol, 2-hexyldecanol, octyldodecanol, dibutyl adipate, medium-chain triglycerides, propylene glycol dicaprylate/dicaprate, propylene glycol laurate, isopropyl myristate, isopropyl palmitate, propylene carbonate, dipropylene glycol monomethyl ether, diethylene glycol monoethyl ether and ketones.

In other embodiments, solvents which have good spreading properties can be used in the formulation, such as 2-hexyldecanol, octyldodecanol, 2-octyldodecyl myristate, cetearyl isononanoate, cetearyl octanoate, cetylethyl hexanoate, cococaprylate/caprate, decyl cocoate, decyl oleate, ethyl oleate, isocetyl palmitate, isopropyl myristate, isopropyl palmitate, isostearyl isostearate, octyl palmitate, octyl stearate, oleyl erucate, medium-chain triglycerides, propylene glycol dicaprylate/dicaprate, dipropylene glycol monomethyl ether, diethylene glycol monoethyl ether, cetyldimethicone, dimethicone and simethicone.

In some embodiments, preference is given to solvents which possess good macrocyclic lactone-dissolving properties and possess good spreading properties, such as 2-hexyldecanol, octyldodecanol, dibutyl adipate, dipropylene glycol monomethyl ether, diethylene glycol monoethyl ether, medium-chain triglycerides, propylene glycol-dicaprylate/dicaprate, propylene glycol laurate, isopropyl myristate and isopropyl palmitate.

These solvents can be employed either alone or in a mixture with additional solvents (cosolvents).

The solvents are present in a concentration of at least about 60 to about 95% by weight, preferably at least about 85 to about 95% by weight.

In other embodiments, the solvents are present in a concentration of at least 65% by weight, at least 70% by weight, at least 75% by weight, at least 80% by weight, at least 81% by weight, at least 82% by weight, at least 83% by weight, at least 84% by weight, at least 85% by weight, at least 86% by weight, at least 87% by weight, at least 88% by weight, at least 89% by weight, at least 90% by weight, at least 91% by weight, at least 92% by weight, at least 93% by weight, at least 94% by weight, at least 95% by weight, at least 96% by weight, at least 97% by weight, at least 98% by weight, at least 99% by weight.

In other embodiments, the solvents are present in a concentration of about 60 to about 95% by weight, about 65 to about 95% by weight, about 70 to about 95% by weight, about 75 to about 95% by weight, about 80 to about 95% by weight, about 81 to about 95% by weight, about 82 to about 95% by weight, about 83 to about 95% by weight, about 84 to about 95% by weight, about 85% to about 95% by weight, about 86 to about 95% by weight, about 87 to about 95% by weight, about 88 to about 95% by weight, about 89 to about 95% by weight, about 90 to about 95% by weight, about 91 to about 95% by weight, about 92 to about 95% by weight, about 93 to about 95% by weight, about 94 to about 95% by weight, about 85% to about 95% by weight, about 85 to about 94% by weight, about 85 to about 93% by weight, about 85 to about 94% by weight, about 85 to about 93% by weight, about 85 to about 92% by weight, about 85 to about 91% by weight, about 85 to about 90% by weight, about 85 to about 89% by weight, about 85 to about 88% by weight, about 85 to about 87% by weight, about 85 to about 86% by weight, about 86 to about 88% by weight, or about 87 to about 89%.

Cosolvents

Suitable additional solvents or cosolvents include cyclic carbonates or lactones. Exemplary cosolvents include, but are not limited to, ethylene carbonate, propylene carbonate, and y-butyrolactone.

The cosolvents are present in a concentration from 5.0 to 60% by weight, preferably from 7.5 to 40% by weight, more preferably from 10 to 20% by weight.

In other embodiments, the cosolvents are present in a concentration of about 11 to about 20% by weight, about 12 to about 20% by weight, about 13 to about 20% by weight, about 14 to about 20% by weight, about 15 to about 20% by weight, about 16 to about 20% by weight, about 17 to about 20% by weight, about 18 to about 20% by weight, about 10 to about 19% by weight, about 10 to about 18% by weight, about 10 to about 17% by weight, about 10 to about 16% by weight, about 10 to about 15% by weight, about 10 to about 14% by weight, about 10 to about 13% by weight, about 10 to about 12% by weight, about 11 to about 13% by weight, about 12 to about 14% by weight, about 13 to about 15% by weight, about 14 to about 16% by weight, about 15 to about 17% by weight, about 16 to about 18% by weight, about 17 to about 19% by weight, or about 18 to about 20% by weight.

The combined concentration of solvent plus cosolvent is generally at least 90% by weight. Preferably, the combined concentration of solvent plus cosolvent is at least 95% by weight. More preferably, the combined concentration of solvent plus cosolvent is at least 97% by weight.

In other embodiments, the combined concentration of solvent plus cosolvent is from about 90 to about 99.9% by weight, from about 91 to about 99.9% by weight, from about 92 to about 99.9% by weight, from about 93 to about 99.9% by weight, from about 94 to about 99.9% by weight, from about 95 to about 99.9% by weight, from about 96% to about 99.9% by weight, from about 97 to about 99.9% by weight, from about 98 to about 99.9% by weight, from about 99 to about 99.9% by weight, from about 90 to about 99.5% by weight, from about 90 to about 99% by weight, from about 90 to about 98% by weight, from about 90 to about 97% by weight, from about 90 to about 96% by weight, from about 90 to about 95% by weight, from about 90 to about 94% by weight, from about 90 to about 93% by weight, from about 90 to about 92% by weight, or from about 90 to about 91% by weight.

Preferably, the combined concentration of solvent plus cosolvent is from about 97 to about 99.9% by weight, from about 97.5 to about 99.9% by weight, from about 98 to about 99.9% by weight, from about 98.5 to about 99.9% by weight, from about 99 to about 99.9% by weight, from about 99.5 to about 99.9% by weight, from about 97 to about 99.5% by weight, from about 97 to about 99% by weight, from about 97 to about 98.5% by weight, or from about 97 to about 98% by weight.

In other embodiments, the combined concentration of solvent plus cosolvent is about 95% by weight, about 95.5% by weight, about 96% by weight, about 96.5% by weight, about 97% by weight, about 97.5% by weight, about 98% by weight, about 98.5% by weight, about 99% by weight, about 99.5% by weight, or about 99.9% by weight.

Adjuvants

The formulations of the invention may optionally contain further adjuvants in a concentration, for example, of from 0.01% to 10% by weight. Preferably, if present, the concentration of the further adjuvants is from 0.025% to 5% by weight. More preferably, if present, the concentration of the further adjuvants is from 0.05 to 1% by weight.

Suitable further adjuvants include: preservatives, thickeners, colorants, spreading oils, antioxidants, light stabilizers, adhesives or tackifiers, emulsifiers, propellants, viscosity-increasing substances and emulsion stabilizers, wetting agents, carriers, lubricants, and glidants.

Preservatives include, for example, benzyl alcohol (unless already present as solvent), trichlorobutanol, p-hydroxybenzoic esters, and n-butanol.

Thickeners include, for example, inorganic thickeners such as bentonites, colloidal silicic acid, aluninium monostearate, organic thickeners such as cellulose derivatives, polyvinyl alcohols, polyvinylpyrrolidones and copolymers thereof, acrylates and methacrylates.

Colorants include all colorants where use on the animal is permitted, which may be dissolved or suspended.

Spreading oils include, for example, di-2-ethylhexyl adipate, isopropyl myristate, dipropylene glycol pelargonate, cyclic and acyclic silicone oils such as dimeticones and also co- and terpolymers thereof with ethylene oxide, propylene oxide and formalin, fatty acid esters, triglycerides, fatty alcohols.

Antioxidants include, for example, sulphites or metabisulphites such as potassium metabisulphite, ascorbic acid, butylated hydroxytoluene (BHT), butylated hydroxyanisole (BHA), tocopherol. The antioxidant, when present, is customarily present in the formulations at concentrations of 0.2% by weight or less, preferably of 0.1% by weight or less.

Light stabilizers include, for example, substances from the class of the benzophenones or novantisol acid.

Adhesives or tackifiers include, for example, cellulose derivatives, starch derivatives, polyacrylates, natural polymers such as alginates, gelatin.

Oils that may be used in emulsions include, for example, paraffin oils, silicone oils, natural vegetable oils such as sesame seed oil, almond oil, castor oil, synthetic triglycerides such as caprylic/capric acid biglyceride, triglyceride mixture with vegetable fatty acids of chain length C₈₋₁₂ or with other specifically selected natural fatty acids, partial glyceride mixtures of saturated or unsaturated fatty acids which may also contain hydroxyl groups, and mono- and diglycerides of the C₈/C₁₀-fatty acids.

Fatty acid esters that can be used in emulsions include, for example, ethyl stearate, di-n-butyryl adipate, hexyl laurate, dipropylene glycol pelargonate, esters of a branched fatty acid of medium chain length with saturated fatty alcohols of chain length C₁₆-C₁₈, isopropyl myristate, isopropyl palmitate, caprylic/capric esters of saturated fatty alcohols of chain length C₁₂-C₁₈, isopropyl stearate, oleyl oleate, decyl oleate, ethyl oleate, ethyl lactate, waxy fatty acid esters such as dibutyl phthalate, diisopropyl adipate, ester mixtures related to the latter, and other fatty alcohols such as isotridecyl alcohol, 2-octyldodecanol, cetylstearyl alcohol and oleyl alcohol.

Fatty acids include, for example, oleic acid and its mixtures.

Emulsifiers include nonionic surfactants, such as polyoxyethylated castor oil, polyoxyethylated sorbitan monooleate, sorbitan monostearate, glycerol monostearate, polyoxyethylstearate, alkylphenol polyglycol ethers;

ampholytic surfactants, such as di-Na N-lauryl-β-ininodipropionate or lecithin;

anionic surfactants, such as Na-lauryl sulphate, fatty alcohol ether sulphates, mono/dialkyl-polyglycol ether orthophosphoric ester monoethanolamine salt; and

cationic surfactants, such as cetyltrimethylammonium chloride.

Further adjuvants are agents with which the formulations according to the invention can be sprayed or squirted onto the skin. These are the conventional propellent gases required for spray cans, such as propane, butane, dimethyl ether, CO₂ or halogenated lower alkanes, or mixtures thereof with one another.

Viscosity-increasing substances and emulsion stabilizers include, for example, carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silica, or mixtures of the substances mentioned.

Carriers include all physiologically acceptable solid inert substances. Suitable as such are inorganic and organic substances. Examples of inorganic substances are sodium chloride, carbonates such as calcium carbonate, hydrogen carbonates, aluminium oxides, silicas, clays, precipitated or colloidal silicon dioxide, and phosphates.

Lubricants and glidants include, for example, magnesium stearate, stearic acid, talc and bentonites.

Endoparasites

While having low toxicity to warm-blooded species, the formulations according to the invention are suitable for combating and/or preventing pathogenic endoparasites that occur in humans and in animal keeping and animal breeding in useful animals, breeding animals, zoo animals, laboratory animals, animals for experimentation and hobby animals (i.e., pets). In this context, they are active against all or individual stages of development of the pests (e.g., larvae, immature adult, adult) and against resistant and normally sensitive species. By combating the pathogenic endoparasites the intention is to reduce disease, mortality and reductions in yield (for example in the production of meat, milk, wool, hides, eggs, honey, etc.), so that the use of the active substances enables more economic and simpler animal keeping.

The pathogenic endoparasites include cestodes, trematodes, nematodes and Acantocephala, in particular:

From the order of the Pseudophyllidea, for example: Diphyllobothrium spp., Spirometra spp., Schistocephalus spp., Ligula spp., Bothridium spp., Diplogonoporus spp.

From the order of the Cyclophyllidea, for example: Mesocestoides spp., Anoplocephala spp., Paranoplocephala spp., Moniezia spp., Thysanosomsa spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.

From the subclass of the Monogenea, for example: Gyrodactylus spp., Dactylogyrus spp., Polystoma spp.

From the subclass of the Digenea, for example: Diplostomum spp., Posthodiplostomum spp., Schistosoma spp., Trichobilharzia spp., Omithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp. Metorchis spp., Heterophyes spp., Metagonimus spp.

From the order of the Enoplida, for example: Trichuris spp. (e.g., Trichuris vulpis), Capillaria spp., Trichomosoides spp., Trichinella spp.

From the order of the Rhabditia, for example: Micronema spp., Strongyloides spp.

From the order of the Strongylida, for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp. (e.g., Ancylostoma caninum), Uncinaria spp. (e.g., Uncinaria stenocephala), Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp., Parelaphostrongylus spp., Crenosoma spp., Paracrenosoma spp., Angiostrongylus spp., Aelurostrongylus spp., Filaroides spp., Parafilaroides spp., Trichostrongylus spp., Haemonchus spp., Ostertagia spp., Marshallagia spp., Cooperia spp., Nematodirus spp., Hyostrongylus spp., Obeliscoides spp., Amidostomum spp., Ollulanus spp.

From the order of the Oxyurida, for example: Oxyuris spp., Enterobius spp., Passalurus spp., Syphacia spp., Aspiculuris spp., Heterakis spp.

From the order of the Ascaridia, for example: Ascaris spp., Toxascaris spp. (Toxascaris leonina), Toxocara spp. (e.g., Toxocara canis), Parascaris spp., Anisakis spp., Ascaridia spp.

From the order of the Spirurida, for example: Gnathostoma spp., Physaloptera spp., Thelazia spp., Gongylonema spp., Habronema spp., Parabronema spp., Draschia spp., Dracunculus spp.

From the order of the Filariida, for example: Stephanofilaria spp., Parafilaria spp., Setaria spp., Loa spp., Dirofilaria spp. (e.g., Dirofilaria immitis), Litomosoides spp., Brugia spp., Wuchereria spp., Onchocerca spp.

From the order of the Gigantorhynchida, for example: Filicollis spp., Moniliformis spp., Macracanthorhynchus spp., Prosthenorchis spp.

The compositions of the invention are advantageously used for the prevention of heartworm disease caused by Dirofilaria spp., such as by Dirofilaria immitis. Prior to administration of the compositions for prevention of heartworm disease, animals are preferably tested for existing heartworm infection. If adult heartworms are detected, the animal is preferably treated with an adulticide effective against Dirofilaria spp. prior to application of the compositions of the invention for the prevention of heartworm disease.

The compositions of the invention are also advantageously used for the treatment and/or control of intestinal parasites (e.g., hookworm, roundworm, and/or whipworm). In some embodiments, the hookworm is Ancylostoma caninum or Uncinaria stenocephala. In some embodiments, the roundworm is Toxocara canis or Toxascaris leonine.

Animals

Productive livestock and breeding animals include mammals, such as, for example, cattle, horses, sheep, pigs, goats, camels, water buffalo, donkeys, rabbits, fallow deer, reindeer, fur-bearing animals, such as, for example, mink, chinchilla or racoon, birds, such as, for example, chickens, geese, turkeys, ducks and ostriches.

Laboratory and experimentation animals include, for example, mice, rats, guinea pigs, golden hamsters, dogs and cats.

Pets include, for example, dogs and cats.

Modes of Administration

Administration can be effected prophylactically as well as therapeutically.

The active compounds are administered, directly or in the form of suitable preparations, dermally, by environment treatment, or with the aid of active-compound-containing shaped articles such as, for example, strips, plates, bands, collars, ear marks, limb bands, marking devices.

Dermal administration is effected, for example, in the form of bathing, dipping, spraying, pouring on, spotting on, washing, shampooing, pouring over, and dusting.

The formulation of the invention can be any suitable form for application to an animal, e.g. an animal's skin. For example, gels, which are applied to, or brushed onto, the skin, can be prepared by treating solutions that have been prepared as described above with such an amount of thickener that a clear substance of cream-like consistency is formed. Thickeners applied are the thickeners indicated further above.

Pour-on and spot-on formulations are poured or sprayed onto limited areas of the skin, the active compound spreading on the body surface.

Pour-on and spot-on formulations are prepared by dissolving, suspending or emulsifying the active compound in suitable solvents or solvent mixtures described above which are tolerated by the skin. If appropriate, other adjuvants such as colorants, antioxidants, light stabilizers and tackifiers are added.

Suitable preparations include:

solutions or concentrates for administration after dilution, solutions for use on the skin, pour-on and spot-on formulations, gels;

emulsions and suspensions for dermal administration, and semi-solid preparations;

formulations in which the active compound is incorporated into a cream base or into an oil-in-water or water-in-oil emulsion base;

solid preparations such as powders, or shaped articles containing active compound.

Solutions for use on the skin are applied dropwise, brushed on, rubbed in, sprayed on, splashed on, or applied by dipping, bathing or washing.

The solutions are prepared by dissolving the active compound in a suitable solvent and, if appropriate, adding additives such as solubilizers, acids, bases, buffer salts, antioxidants and preservatives.

According to the invention, particular preference is given to pour-on or spot-on formulations. These are applied in comparatively small quantities of what is usually from 0.1 to 20 ml, preferably of from 0.4 to 10 ml, to a small part of the body surface of the animal to be treated.

In a preferred embodiment, the pour-on or spot-on formulations are packaged as ready-to-use solutions in single dose applicator tubes.

The solvents which are suitable for the pour-on or spot-on formulations are those which are mentioned above.

Spot-on or pour-on formulations can also be formulated as emulsion concentrates. In this connection, the active compounds are dissolved, at elevated concentration, in a solvent together with a dispersant. The user adds a given quantity of this concentrate to water, whereupon an emulsion forms either spontaneously or after shaking. The above-mentioned substances can be used as solvents while the ionic and nonionic emulsifiers which are likewise mentioned above can be used as dispersants.

Emulsions are either of the water-in-oil type or of the oil-in-water type. They are prepared by dissolving the active compound either in the hydrophobic or in the hydrophilic phase and homogenizing this phase with the solvent of the other phase, with the aid of suitable emulsifiers and, if appropriate, other adjuvants such as colorants, absorption accelerators, preservatives, antioxidants, light stabilizers, viscosity-increasing substances.

Suspensions are prepared by suspending the active compound in an excipient liquid, if appropriate with an addition of further adjuvants such as wetting agents, colorants, absorption accelerators, preservatives, antioxidants and light stabilizers.

Semi-solid preparations for dermal administration are only distinguished from the above-described suspensions and emulsions by their higher viscosities.

To prepare solid preparations, the active compound is mixed with suitable carriers, if appropriate with the addition of adjuvants, and the mixture is formulated as desired.

Formulations of the invention have been demonstrated as safe to use in animals concomitantly receiving ACE inhibitors, anticonvulsants, antihistamines, antimicrobials, chondroprotectants, corticosteroids, immunotherapeutics, MAO inhibitors, NSAIDs, ophthalmic medications, sympathomimetics, synthetic estrogens, thyroid hormones, and urinary acidifiers.

### EXAMPLES—SPOT ON TREATMENT FOR DOGS

### Example 1

| | |
|---|---|
| Moxidectin | 2.50 g |
| Benzyl Alcohol | 87.79 g |
| Propylene Carbonate | 16.50 g |
| Butylhydroxytoluene (BHT) | 0.10 g |

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1. A parasiticidal formulation comprising about 0.1 to about 10% by weight of one or more macrocyclic lactones, about 60 to about 95% by weight of one or more solvents selected from the group consisting of benzyl alcohols and substituted pyrrolidones, and about 5 to about 60% by weight of one or more cosolvents selected from the group consisting of cyclic carbonates and lactones, wherein the one or more macrocyclic lactones are the sole parasiticidal active compound.

2. The parasiticidal formulation of claim 1 comprising about 0.5 to about 5% by weight of one or more macrocyclic lactones, about 75 to about 95% by weight of one or more solvents selected from the group consisting of benzyl alcohols and substituted pyrrolidones, and about 10 to about 20% by weight of one or more cosolvents selected from the group consisting of cyclic carbonates and lactones.

3. The parasiticidal formulation of claim 1 further comprising an adjuvant selected from the group consisting of: a preservative, a thickener, a colorant, an antioxidant, a light stabilizer, a tackifier, and combinations thereof.

4. The parasiticidal formulation of claim 3, wherein the adjuvant is an antioxidant.

5. The parasiticidal formulation of claim 3, wherein the adjuvant is butylated hydroxytoluene (BHT).

6. The parasiticidal formulation of claim 1, wherein the macrocyclic lactone is selected from the group consisting of: avermectin B₁ₐ/B_{1b}, 22,23-dihydroavermectin (B₁ₐ/B_{1b}), doramectin, moxidectin, selamectin, and eprinomectin.

7. The parasiticidal formulation of claim 1, wherein the macrocyclic lactone is moxidectin.

8. The parasiticidal formulation of claim 1, wherein the one or more solvents is benzyl alcohol.

9. The parasiticidal formulation of claim 1, wherein the one or more cosolvents is propylene carbonate.

10. The parasiticidal formulation of claim 1, wherein the macrocyclic lactone is moxidectin, wherein the solvent is benzyl alcohol, and wherein the cosolvent is propylene carbonate.

11. The parasiticidal formulation of claim 10, wherein benzyl alcohol is the sole solvent and wherein propylene carbonate is the sole cosolvent.

12. The parasiticidal formulation of claim 1, wherein the formulation is a spot-on formulation.

13. The parasiticidal formulation of claim 2 comprising about 80% to about 95% by weight of one or more solvents.

14-24. (canceled)

25. A method of controlling endoparasites in an animal comprising applying to the animal the parasiticidal formulation of claim 1.

26. The method of claim 25, wherein the animal is selected from the group consisting of, a human, a livestock animal, and a pet.

27. The method of claim 25, wherein the animal is a dog or a cat.

28. The method of claim 25, wherein the formulation is applied to the skin of the animal.

29. (canceled)

30. A method of controlling endoparasites in an animal comprising applying to the animal the parasiticidal formulation of claim 2.

31. A method of controlling endoparasites in an animal comprising applying to the animal the parasiticidal formulation of claim 13.
